(19) United States
(12) Patent Application Publication
(10) Pub. No.: US 2017/0166638 A1
(43) Pub. Date: Jun. 15, 2017

(54) USE OF CD-64-BLOCKING AGENTS AS ENHANCER OF AN ANTI-TNF-a ANTIBODY THERAPY

(71) Applicant: Fraunhofer-Gesellschaft zur Foerderung der angewandten Forschung e.V., Munich (DE)

(72) Inventors: Dmitrij HRISTODOROV, Aachen (DE); Theophilus THEPEN, Aachen (DE); Stefan BARTH, Aachen (DE)

(73) Assignee: Fraunhofer-Gesellschaft zur Foerderung der angewandten Forschung e.V., Munich (DE)

(21) Appl. No.: 15/126,690

(22) PCT Filed: Mar. 26, 2015
(86) PCT No.: PCT/EP2015/056622

§ 371 Date: Sep. 16, 2016

(30) Foreign Application Priority Data
Mar. 26, 2014 (EP) 14161731.6

(51) Int. Cl. C07K 16/28; C07K 16/24; A61K 39/395

(52) U.S. Cl. CPC C07K 16/283; A61K 39/3955; C07K 16/241; C07K 2317/92; A61K 2039/507

(57) ABSTRACT

A polypeptide having a binding affinity to CD64 of at least 10⁻⁹ M for use in a treatment of a disease associated with an inflammation in combination with an anti-TNF-α, antibody.

The present invention pertains to a polypeptide for use in a treatment of a disease associated with an inflammation in patients not responding to a treatment of inflammation by a therapy employing an anti-TNF-α antibody.

Tumor necrosis factor alpha (TNF-α) is a key cytokine involved in the pathogenesis of several inflammatory diseases, including inflammatory bowel disease (IBD), rheumatoid arthritis (RA), and atopic dermatitis (AD) (Roberts-Thomson, et al., Cells, cytokines and inflammatory bowel disease: a clinical perspective. Expert Rev Gastroenterol Hepatol 2011, 5: 703-716; Upchurch, K. S. and Kay, J., Evolution of treatment for rheumatoid arthritis. Rheumatology (Oxford) 2012, 51; Numerof, R. P. and Asadullah, K., Cytokine and anti-cytokine therapies for psoriasis and atopic dermatitis. BioDrugs 2006, 20: 93-103). It is initially produced as a transmembrane protein (mTNF-α), which is cleaved off by the membrane-resident protease ADAM17 to yield the soluble form (Black, R. A., et al., A metalloproteinase disintegrin that releases tumour-necrosis factor-alpha from cells, Nature 1997, 385: 729-733).

Both forms can bind to one of the two known TNF receptors (TNFR1 or TNFR2) thus activating NF-κB and AP-1 (Liu, Z. G., Molecular mechanism of TNF signaling and beyond. Cell Res 2005. 15: 24-27.), which are regulators of pro-inflammatory genes including those encoding cytokines such as IL-1β, IL-6, IL-8, GM-CSF and TNF-α itself (Rogler, G. and Andus, T., Cytokines in inflammatory bowel disease. World J Surg 1998, 22: 382-389).

The development of monoclonal antibodies (mAbs) that neutralize soluble TNF-α has led to a number of successful therapies that interrupt the downstream cascade of pro-inflammatory events (Thalayasingam, N. and Isaacs, J. D., Anti-TNF therapy. Best Pract Res Clin Rheumatol 2011. 25: 549-567). However, some patients do not respond to anti-TNF-α therapy. Recently an increased expression of CD64 on monocytes and macrophages in non-responding IBD patients was reported (Wojtal, K. A., et al., Fc gamma receptor CD64 modulates the inhibitory activity of infliximab. PLoS One 2012. 7: e43361). Infliximab and adalimumab both prominent anti-TNF-α mAbs are captured by CD64 inducings a pro-inflammatory downstream signaling cascade, which counteracts the positive effects achieved by neutralizing TNF-α. Although it has been reported that incubation with purified IgG1 Fcγ domains improved the efficacy of these mAbs under laboratory conditions (Bruhns, P., et al., Specificity and affinity of human Fcgamma receptors and their polymorphic variants for human IgG subclasses. Blood 2009. 113: 3716-3725), However, this approach is unsuitable in the clinic because low-affinity Fcγ receptors CD32 and CD16 become blocked, which induces immunosuppression by inhibiting antibody dependent cellular cytotoxicity (ADCC) and complement dependent cytotoxicity (CDC), CD32 and CD16 are necessary to mediate effector functions, including ADCC and CDC, thus providing essential protection against invading pathogens. Blocking all three Fcγ receptors would suppress the immune system and leave patients susceptible to opportunistic infections (Willcocks, L. C., et al., Low-affinity Fcgamma receptors, autoimmunity and infection, Expert Rev Mol Med 2009. 13; and Nimmerjahn, F. and Ravetch, J. V., Fc-receptors as regulators of immunity. Adv Immunol 2007. 96: 179-204).This is a particular concern because anti-TNF-α therapy can already is induce moderate immunosuppression, so further compromising the immune system would be highly undesirable (Ford, A. C. and Peyrin-Biroulet, L., Opportunistic infections with anti-tumor necrosis factor-alpha therapy in inflammatory bowel disease: meta-analysis of randomized controlled trials. Am J Gastroenterol 2013. 108: 1268-1276; Xie, X., Li, F., et al., Risk of tuberculosis infection in anti-TNF-alpha biological therapy: From bench to bedside. J Microbiol Immunol Infect 2013. 30: 005; and Ordonez, M. E., Farraye, F. A. and Di Palma, J. A., Endemic Fungal Infections in Inflammatory Bowel Disease Associated with Anti-TNF Antibody Therapy, Inflamm Bowel Dis 2013. 19: 2490-2500).

WO 2014/029890A1 discloses a method for predicting the likelihood of a patient being non-responsive to therapy by TNF-alpha antibodies, wherein in a sample representative of an affected tissue, the expression level of CD64 and/or CD16 are determined and compared to a standard. Similarly, a pharmaceutical composition for treating an autoimmune disease is disclosed, wherein the composition comprises a TNF alpha antibody and an antibody against CD64 and/or CD16, or a non-specific antibody preparation.

WO 2005/052007A1relates to a heterologous, recombinantly prepared complex which comprises at least one cytotoxic domain and at least one CD64-specific binding domain, especially of human origin, and nucleic acids and vectors coding for such a complex. It further relates to methods for influencing cell growth and the physiology of CD64-positive cells with the complex according to the invention or with vectors containing the nucleic acid coding therefore. The invention further relates to vectors and hosts for producing the complex according to the invention. It further relates to the preparation and distribution of medicaments based on the complex according to the invention or vectors coding therefore, for the treatment of diseases based on a pathological proliferation and/or increased activity of structurally defined cell populations. This applies, in particular, to tumour diseases, allergies, autoimmune diseases, infectious diseases, chronic inflammation or transplantations (immune suppression).

WO 2006/002438 A2 discloses isolated monoclonal antibodies, particularly human antibodies, that bind to CD64 with high affinity. Nucleic acid molecules encoding the antibodies of the invention, expression vectors, host cells and methods for expressing the antibodies of as well as immunoconjugates, bispecific molecules and pharmaceutical compositions comprising the antibodies of the invention are also disclosed. The disclosure also provides methods for treating autoimmune disorders, transplant rejection, Graft Versus Host Disease, or cancer and for enhanced presentation of antigen using conjugates of an antigen and an anti-CD64 antibody.

Randall T. Curnow reports about clinical experience with CD64-directed immuno-therapy in an overview article in Cancer Immunology, Immunotherapy, November 1997, Volume 45, Issue 3-4, pp 210-215. The overview summarizes the clinical experience with CD64-directed immunotherapy in cancer patients with the bispecific antibodies MDX-447 [humanized Fab anti-CD64×humanized Fab anti-(epidermal growth factor receptor, EGFR)] and MDX-H210 (humanized Fab anti-DC64 x Fab anti-HER2/neu), and with the anti-CD64 monoclonal antibody (mAB) MDX-33 (H22) in the modulation of monocyte CD64 in vivo.

### BRIEF DESCRIPTION OF THE INVENTION

An object of the present invention is to provide a method for enhancing the therapy with anti-TNF-α antibodies.

Another object is to provide a method for treatment of patients who are non-responders in a treatment of inflammatory diseases by anti-TNF-α antibodies.

Still another object of the present invention is to provide a compound for use in enhancing the therapy with anti-TNF-α antibodies.

Surprisingly these objects are accomplished by a polypeptide having a binding affinity to CD64 of at least 10⁻⁹ M. According to the invention this polypeptide can be used in a treatment of a disease associated with an inflammation in combination with an anti-TNF-α antibody.

The polypeptide of the present invention can be administered to patients not responding to a treatment of inflammation by a therapy employing an anti-TNF-α antibody or to patients whose therapy has not yet started and were not exposed before to an anti-TNF-α antibody.

### BRIEF DESCRIPTION OF THE DRAWINGS

FIG. 1 upper panel graphically depicts the results of flow cytometry experiments showing the effects of IgG4 in reducing the capture of anti-TNF-α mAbs as mediated by CD16.

FIG. 1 lower panel graphically depicts the results of flow cytometry experiments showing the effects of human aglycoIgG1 mAbs in not reducing the capture of anti-TNF-α mAbs.

FIG. 2 graphically depicts the results of flow cytometry experiments showing that neither IgG4 nor aglycoIgG1 bind to CD64 in the absence of serum.

FIG. 3(a) depicts a representation of coding sequence H22(scFv) fused in frame with an N-terminal pelf leader peptide and a His₁₀ tag.

FIG. 3(b) pholostatically depicts results of molecular weight analysis by SDS-PAGE and by western blot, which confirm the identity of purified H22(scFv).

FIG. 3(c) graphically depicts the results of flow cytometry experiments showing that H22(scFv) specifically binds to CD64⁺ target cells.

FIG. 4(a) graphically depicts the results of flow cytometry experiments showing that H22(scFv) is able to block CD64.

FIG. 4(b) graphically depicts the results of a flow cytometry experiment showing the H22(scFv) reduces the capture of anti-TNF-α mAbs.

FIG. 5(a) graphically depicts the results of measuring the concentration of ant-CD64 mAb required to saturate all CD64 molecules on the cell surface.

FIG. 5(b) graphically depicts the results of directly titrating H22(scFv) against cultured HL-60 cells.

FIG. 5(c) graphically depicts the results of experiments showing that blocking CD64 with H22(scFv) quantitatively correlates with reduced capture of anti-TNF-α mAbs,

FIG. 6 graphically depicts the results of inducing cytokine gene expression measured by real-time PCR using gene-specific primers.

FIG. 7 graphically depicts the results of flow cytometry experiments showing that HL-60 cells only when stimulated express mTNF-α.

FIGS. 8(a)-(c) graphically depict the results of flow cytometry experiments showing that blocking mTNF-o reduces the ability of HL-60 cells to capture anti-TNF-α mAbs.

FIG. 8(d) graphically depicts the results of experiments wherein the additive effect of blocking by H22(scFv) and anti-mTNF-α is found to match the total capacity of stimulated HL-60 cells to capture anti-TNF-α mAbs.

FIG. 9 schematically depicts that blocking both CD64 and mTNF-α genes completely abolishes the ability of stimulated HL-60 cells to capture anti-TNF-α mAbs.

In particular embodiment of the invention, the polypeptide for the use according to the invention wherein the disease associated with an inflammation, is selected from the group consisting of inflammatory bowel disease, rheumatoid arthritis, multiple sclerosis, lupus, sclerodoma; psoriasis; gastrointestinal, e.g., graft-versus-host-disease, Crohn's disease, diabetes, ulcerative colitis, acute and chronic transplantation rejection, colitis, alveolitis, bronchiolitis obliterans, ileitis, pancreatitis, glomerulonephritis, uveitis, arthritis, hepatitis, dermatitis, artherosclerosis, all forms of arthritis, including, but not only Rheumatoid arthritis, Multiple sclerosis, ankylosing spondylitis, hidradenitis suppurativa and atopic dermatitis.

In a particular embodiment the polypeptide is an antibody or an antibody fragment such as a scFv fragment, in particular the fragment H22(scFv), represented by the polypeptide comprising or consisting of the amino acid sequence encoded by the polynucleotide of Seq ID No. 1.

In a further embodiment of the invention the H22(scFv) compound is a recombinant polypeptide having the SEQ ID No 2.

HHHHHHHHHHSSGHIDDDDKHMKLMAQPAMAQVQLVESGGGVVQPGRSLR
LSCSSSGFIFSDNYMYWVRQAPGKGLEWVATISDGGSYTYYPDSVKGRFT
ISRDNSKNTLFLQMDSLRPEDTGVYFCARGYYRYEGAMDYWGQGTPVTVS
SGGGGSGGGGSGGGGSDIQLTQSPSSLSASVGDRVTITCKSSQSVLYSSN
QKNYLAWYQQKPGKAPKLLIYWASTPESGVPSRFSGSGSGTLFIFTISSL
QPEDIATYYCHQYLSSWTFGQGTKLEIK

Subject matter of the invention is also a mixture comprising the polypeptide of the invention and a blocking agent of transmembrane TNF-α, such as antibodies or derivatives thereof, siRNAs/anti-sense RNAs reducing the expression level of membrane TNF-alpha.

Subject matter of the invention is also a method of treating a disease associated with an inflammation by administering to a patient in need thereof a polypeptide of the invention in combination with an anti-TNF-α antibody.

In an embodiment of the invention the patient is a non-responder to a treatment of inflammation in a therapy employing an anti-TNF-α antibody.

A further embodiment of the invention is a method for enhancing the therapy of a patient in a therapy employing an anti-TNF-α antibody, in particular of a patient who is a non-responder to a treatment of inflammation in a therapy employing an anti-TNF-α antibody,

In yet another embodiment of the method of the invention the polypeptide of the invention is administered in combination with a blocking agent of transmembrane TNF-α.

### DETAILED DESCRIPTION OF THE INVENTION

In order to solve the technical problem underlying the present invention it was aimed to develop a CD64-specific therapeutic candidate to prevent the capture of the Fcγ domain.

Although replacing the Fcγ domain of human IgG1 with its IgG4 counterpart would be a choice, because IgG4 binds CD64 with >100-fold greater affinity than CD32 or CD16 (Bruhns, P., Iannascoli, B., England, P., Mancardi, D. A., Fernandez, N., Jorieux, S. and Daeron, M., Specificity and affinity of human Fcgamma receptors and their polymorphic variants for human IgG subclasses. Blood 2009. 113: 3716-3725), this resulted in the finding that IgG4 was able to reduce the capture of anti-TNF-α mAbs by stimulated HL-60 cells, but surprisingly the effect was mediated by blocking CD16 rather than CD64, as initially expected. This may reflect the greater abundance of CD16 compared to CD64 on the surface of stimulated HL-60 cells. Although these in vitro results confirmed the involvement of CD16 in the capture of anti-TNF-α mAbs and agree with previous observations, IgG4 nevertheless proved unsuitable for the selective blocking of CD64.

Genetically and enzymatically aglycosylated forms of the IgG1-Fcγ domain are known to lose their ability to bind CD16 and CD32, but to retain residual binding affinity towards CD64 (Jung, S. T., Kang, T. H., Kelton, W. and Georgiou, G., By-passing glycosylation: engineering aglycosylated full-length IgG antibodies for human therapy. Curr Opin Biotechnol 2011, 22: 858-867). However, in direct contrast to earlier reports, it has been found that aglycoIgG1 blocked CD32, but not CD64 or CD16, Blocking CD32 did not reduce the capacity of HL-60 cells to capture anti-TNF-α mAbs, which indicates that CD32 does not influence the inability of patients to respond to anti-TNF-α therapy,

CD64-specific blocking was ultimately achieved by designing and expressing a recombinant CD64-specific antibody fragment, originally derived from the murine full-length mAb22 (Anderson, C. L., Guyre, P. M., Whitin, J. C., Ryan, D. H., Looney, R. J. and Fanger, M. W., Monoclonal antibodies to Fc receptors for IgG on human mononuclear phagocytes. Antibody characterization and induction of superoxide production in a monocyte cell line. J Biol Chem 1986. 261: 12856-12864). In the course of development, mAb22 was humanized to yield the full-length mAb H22 (Graziano, R. F., et al., Construction and characterization of a humanized anti-gamma-Ig receptor type I (Fc gamma RI) monoclonal antibody. J Immunol 1995. 155: 4996-5002), and then reformatted as a single-chain fragment to yield H22(scFv). We produced recombinant H22(scFv) in E. coli, using a stress-induced periplasmic protein expression protocol that is well-established in our laboratory (Barth, S., et al., Compatible-solute-supported periplasmic expression of functional recombinant proteins under stress conditions. Appl Environ Microbiol 2000. 66: 1572-1579). The addition of recombinant H22(scFv) to stimulated HL-60 cells resulted in the potent and specific blocking of CD64, thus strongly reducing the ability of the cells to capture anti-TNF-α mAbs. This result was unexpected because the full-length mAb22 and H22 have been shown to bind an epitope on CD64 that lies outside of the Fcγ domain binding site (Guyre, P. M., et al., Monoclonal antibodies that bind to distinct epitopes on Fc gamma RI are able to trigger receptor function. J Immunol 1989. 143: 1650-1655). However, despite binding outside of the Fcγ domain binding site, H22 binding has been shown to be independent of the presence of other IgG molecules in human serum (Graziano, R. F., et al., Construction and characterization of a humanized anti-gamma-Ig receptor type I (Fc gamma RI) monoclonal antibody. J Immunol 1995. 155: 4996-5002). In addition, H22 effectively blocks CD64-meditated phagocytosis of opsonized red blood cells and strongly down-regulates the expression of CD64 under physiological conditions (Wallace, P. K., et al., Humanized mAb H22 binds the human high affinity Fc receptor for IgG (FcgammaRI), blocks phagocytosis, and modulates receptor expression. J Leukoc Biol 1997. 62: 469-479). Whether or not the blocking effect of H22(scFv) can be explained by steric hindrance remains unclear; however, all the above mentioned features of this unique anti-CD64 antibody underline its ability to bind, block, and to reduce the surface level of CD64, even under serum conditions. This makes 1-122(scFv) a particular interesting therapeutic candidate for chronic inflammatory diseases.

In addition the treatment according to the invention is further improved by a combined administration of the polypeptide of the invention and blocking agent of transmembrane TNF-α.

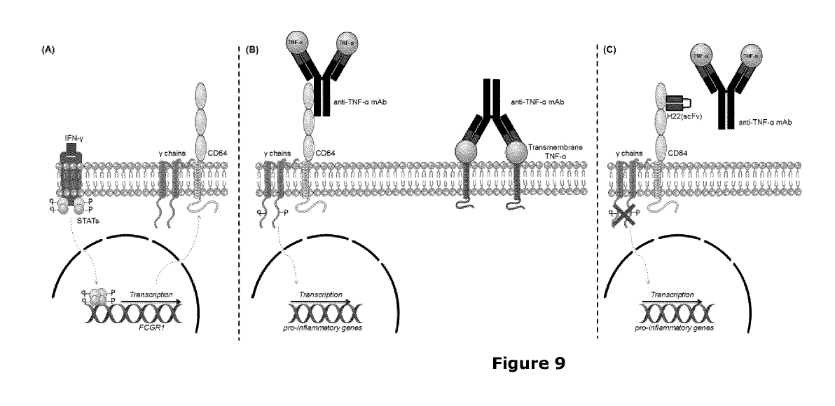

Interestingly, the cytokine TNF-α exists in both soluble and membrane-bound forms. Although the soluble form is pathological under chronic inflammatory conditions and is the therapeutic target of anti-TNF-α mAbs, the role of mTNF-α is not well understood. The inventors examined the surface expression of mTNF-α solely on stimulated (pro-inflammatory) HL-60 cells. It has been found that blocking both CD64 and mTNF-α completely abolished the ability of stimulated HL-60 cells to capture anti-TNF-α mAbs, indicating that both CD64 and mTNF-α have a significant impact on the success of anti-TNF-α therapy (FIG. 9).

In conclusion, CD64-blocking antibodies, in particular the CD64-blocking anti-body fragment H22(scFv), prevent the capture of anti-INF-α mAbs by CD64, whose overexpression on the surface of inflammatory monocytes and macrophages is associated with the poor response of some patients towards anti-TNF-α therapy. CD64-blocking antibodies, in particular the CD64-blocking antibody fragment H22(scFv) promote the therapeutic efficacy of anti-TNF-α mAbs by potentiating their activity. The present invention is also based on the fining that an increased expression of mTNF-α contributes to the capture of anti-TNF-α mAbs via their Fab domain. CD64-blocking antibodies in combination with antibodies that specifically block mTNF-α therefore enhance the efficiency of anti-TNF-α therapy even further and reduce the population of non-responders accordingly,

The polypeptide of the invention can be derivatized, especially at the N terminal, C terminal and/or in the peptide chain. The derivatization of the N terminal may include partial or complete alkylation, acylation or another N modification. The derivatization of the C terminal may include amidation, esterification or another modification of the terminal carboxy group. The derivatization of the polypeptide chain may include, in particular, a modification to improve the properties of the polypeptide according to the invention, for example, PEGylation, HESylation or the like. If desired, the polypeptide according to the invention may also be coupled to structural units having binding affinity for cellular structures of the organism against which the polypeptide according to the invention is to be employed, Suitable derivatizations for achieving modified properties of polypeptides are known to the skilled person. Derivatives of the polypeptide according to the invention having the mentioned activities are also claimed according to the invention.

The polypeptide of the invention shows in particular at least 90% identity to the polypeptide sequences of the polypeptide of the invention.

Thus, according to the invention polypeptides are embodiments of the invention which result from truncation of the sequence or the addition or deletion of further amino acid residues. Further, it is possible to employ variants of the polypeptide that have additional sequence elements having been added, for example, in order to achieve a higher yield in the preparation in a recombinant form, or a facilitated purification.

It may be advantageous to remove individual amino acid residues, or replace them by the residues of different amino acids. The insertion of additional amino acid residues is also possible. Such changes are also possible when proceeding from truncated or extended variants of the polypeptide. In particular, it may be advantageous to replace individual amino acid residues by those having similar physico-chemical properties. Thus, mainly residues of the amino acids are mutually interchangeable within the following groups:

arginine and lysine;

glutamic acid and aspartic acid;

glutamine, asparagine and threonine;

glycine, alanine and proline;

leucine, isoleucine and valine;

tyrosine, phenylalanine and tryptophan;

serine and threonine,

Peptides are formulated as medicaments in various ways which are known to the skilled person. Pharmaceutically acceptable excipients may be present in such medicaments. Pharmaceutically acceptable excipients suitable for use as carriers or diluents are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R, Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995).

A therapeutically effective amount of the polypeptide of the invention in the present medicament may be readily ascertained by one of ordinary skill in the art using publicly available materials and procedures. For example, the amount of the polypeptide can be present in the formulation in an amount of between about 0.1 mg/mL to about 100 mg/mL. In some embodiments, the polypeptide of the invention is present at a concentration of about 1 mg/mL. In some embodiments, the polypeptide of the invention is present at a concentration of about 10 mg/mL. In some embodiments, the polypeptide of the invention is present at a concentration of about 20 mg/mL. In some embodiments, the polypeptide of the invention is present at a concentration of about 25 mg/mL. In some embodiments, the polypeptide of the invention is present at a concentration of about 30 mg/mL. In some embodiments, the polypeptide of the invention is present at a concentration of about 50 mg/mL. In some embodiments, the polypeptide of the invention is present at a concentration of about 75 mg/mL. In some embodiments, the polypeptide of the invention is present at a concentration of about 80 mg/mL. In some embodiments, the polypeptide of the invention is present at a concentration of about 85 mg/mL. In some embodiments, the polypeptide of the invention is present at a concentration of about 90 mg/mL. In some embodiments, the polypeptide of the invention is present at a concentration of about 100 mg/mL,

In various embodiments of the present disclosure, the pharmaceutical formulations have a pH that is obtained by neutralization of the formulations. As used herein, the term “neutralization” means making a composition to have a more neutral pH (i.e., changing the pH of a composition to a pH of about 6.8 to about 7.5). For example, addition of an acidic substance to a basic composition can make the basic composition to have a more neutral pH (i.e., approximately 6.8-7.5). Likewise, addition of a basic substance to an acidic composition can make the acidic composition to have a more neutral pH (i.e., approximately 6.8-7.5).

In some embodiments of the present disclosure, the pH of the pharmaceutical formulations is obtained by neutralizing the formulation after combining the polypeptide of the invention and the one or more excipients. In one embodiment, the neutralization is achieved by addition of the polypeptide of the invention. In another embodiment, the neutralization is achieved by addition of a pharmaceutically acceptable acid or base. In one embodiment, the pharmaceutically acceptable acid or base is sodium hydroxide. In another embodiment, the pharmaceutically acceptable acid or base is sodium acetate. In yet another embodiment, the pharmaceutically acceptable acid or base is sodium citrate. In another embodiment, the pharmaceutically acceptable acid or base is sodium benzoate.

In various embodiments of the present disclosure, the polypeptide of the invention is prepared via lyophilization. The term “lyophilization,” also known as freeze-drying, is a commonly employed technique for presenting proteins which serves to remove water from the protein preparation of interest. Lyophilization is a process by which the material to be dried is first frozen and then the ice or frozen solvent is removed by sublimation in a vacuum environment.

In other embodiments of the present disclosure, a process for preparing a polypeptide of the invention formulation is described. The process includes the step of combining polypeptide of the invention and one or more excipients, wherein the formulation is neutralized to a pH of greater than about 6.8, wherein a range of 6.8-8.0 is acceptable, subsequent to the combination. The previously described embodiments of the pharmaceutical formulations, including excipients, range of pH and specific pH-values, and neutralization techniques are applicable to the process of preparing the formulations,

In other embodiments of the present disclosure, a product made by the process for preparing a polypeptide of the invention formulation is described. The product can be made by the process that includes the step of combining polypeptide of the invention and one or more excipients, wherein the formulation is neutralized to a pH of greater than about 6.8, wherein a range of 6.8-8.0 is acceptable, subsequent to combination. The previously described embodiments of the pharmaceutical formulations, including excipients, range of pH and specific pHs, and neutralization techniques are applicable to the product made by the process of preparing the formulations.

According to the present disclosure, a formulation containing a polypeptide of the invention may be administered by any conventional route suitable for proteins or peptides, including, but not limited to, parenterally, e.g. injections including, but not limited to, subcutaneously or intravenously or any other form of injections or infusions. Formulations containing a polypeptide of the invention can be administered by a number of routes including, but not limited to oral, intravenous, intraperitoneal, intramuscular, transdermal, subcutaneous, topical, sublingual, intravascular, intramammary, or rectal means. Formulations containing a polypeptide of the invention can also be administered via liposomes. Such administration routes and appropriate formulations are generally known to those of skill in the art. Formulations containing a polypeptide of the invention, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations containing a polypeptide of the invention suitable for parenteral administration (e.g., administration via intraarticular, intravenous, intramuscular, intradermal intraperitoneal, and subcutaneous routes) include aqueous and nonaqueous, isotonic sterile injection solutions (which can contain antioxidants, buffers, bacteriostats, and solutes that render the formulation isotonic with the blood of the intended recipient), and aqueous and non-aqueous sterile suspensions (that can include suspending agents, solubilizers, thickening agents, stabilizers, and preservatives). The formulations containing a polypeptide of the invention can be presented in unit-dose or multi-dose sealed containers, such as ampules and vials. The formulations containing a polypeptide of the invention can also be presented in syringes, such as prefilled syringes.

The invention is further explained by the following non limiting examples.

In contrast to the fully human (adalimumab) or the chimeric (infliximab) anti-TNF-α mAb used in the clinics, a mouse-derived anti-human TNF-α mAb due to availability issues has been used. Mouse IgG1-Fcγ has been reported to have a low affinity towards human CD64, which would significantly affect the experimental set-up. However, in contrast to the data found in literature, the here used mouse-anti-human TNF-α mAb could be strongly captured by human CD64 (and also CD16 and CD32) in the experimental cell-based in vitro system, allowing to study the effects of CD64-blocking molecules on the capture of the anti-TNF-α.

Materials and Methods

Antibodies and Cytokines

Antibodies against CD16, CD32, CD64, and TNF-α were purchased from eBioscience (Frankfurt, Germany). Human anti-IgG4 was obtained from AbD Serotec (Duesseldorf, Germany), Human aglycoIgG1 was produced in-house, Anti-mTNF-α (Gerspach, J., et al, Detection of membrane-bound tumor necrosis factor (TNF): an analysis of TNF-specific reagents. Microsc Res Tech 2000, 50: 243-250) was purchased from Hycultec GmbH (Beutelsbach, Germany). Mouse-anti-His₅ and mouse-anti-His,-Alexa Fluor 488 were obtained from Qiagen (Hilden, Germany), Alkaline phosphatase-conjugated anti-mouse-IgG was obtained from Sigma (Steinheim, Germany). Goat-anti-mouse-phycoerythrin (GAM-PE) was from Dianova (Hamburg, Germany). Human IFN-γ and human TNF-α were obtained from Peprotech (Hamburg, Germany) and eBioscience, respectively.

Generation of H22(scFv)

The open reading frame for H22(scFv) was inserted into the bacterial expression vector pMT (Matthey, B., et al., A new series of pET-derived vectors for high efficiency expression of Pseudomonas exotoxin-based fusion proteins. Gene 1999. 229: 145-153) in frame with a pe/B leader peptide sequence and the purification is and detection tag His₁₀ (Hristodorov, et al, Microtubule-associated protein tau facilitates the targeted killing of proliferating cancer cells in vitro and in a xenograft mouse tumour model in vivo. British Journal of Cancer 2013: 1-9). Successful cloning was confirmed by test digestion and sequencing. Expression, purification, and protein analysis were carried out as previously described (Hristodorov, et al, Microtubule-associated protein tau facilitates the targeted killing of proliferating cancer cells in vitro and in a xenograft mouse tumour model in vivo. British Journal of Cancer 2013: 1-9; Barth, S., et al., Compatible-solute-supported periplasmic expression of functional recombinant proteins under stress conditions. Appl Environ Microbiol 2000. 66: 1572-1579).

Cell Culture

The human pro-myelocytic HL-60 cell line (ATCC: CCL-240) and the human Hodgkin lymphoma cell line L540cy (DSMZ: ACC 72) were cultivated in RPMI 1640 medium supplemented with 10% (v/v) fetal calf serum, 2 mM L-glutamine, 50 μg/ml penicillin and 100 μg/ml streptomycin at 37° C., 100% humidity, and 5% CO₂. HL-60 cells were stimulated with 50 U/ml IFN-γ 24 h before each experiment.

Cell-Binding Analysis by Flow Cytometry

The cell-binding activity of each antibody was analyzed by flow cytometry. We incubated 4×10⁵ cells with known amounts of antibody in PBS (pH 7.4) containing 2 mM EDTA and 0.5% (w/v) BSA for 30 min on ice followed by washing with PBS. Unconjugated primary antibodies were detected using an anti-His₅-Alexa Fluor 488 (1:100) or GAM-PE (1:100) for 30 min on ice in the dark. The cells were then washed twice with PBS and analyzed on a FACS Calibur flow cytometer (Becton Dickinson, Heidelberg, Germany).

RNA Isolation, cDNA Synthesis, and Real-Time PCR Analysis

RNA was isolated using SV Total RNA Isolation System (Promega, Mannheim, Germany). Complementary DNA (cDNA) was produced using QuantiTect Reverse Transcription Kit (Qiagen). Real-time PCR was performed using SYBR Green PCR Kit (Qiagen). The cycle conditions were: stage 1 (50° C., 2 min); stage 2 (95° C., 10 min); stage 3: 35 repeats (95° C., 15 s; 60° C., 1 min). The expression levels of the target genes were normalized to the expression levels of GAPDH using the comparative threshold cycle method. The following primers were used: IL-1β_f: ACAGATGAAGTGCTCCTTCCA, IL-1β_r; GTCGGAGATTCGTAGCTGGAT, IL-6_f: GGTACATCCTCGACGGCATCT, IL-6_r: GTGCCTCTTTGCTGCTTTCAC, IL-8_f: ACTGAGAGTGATTGAGAGTGGAC, IL-8_r: AACCCTCTGCACCCAGTTTTC, GM-CSF_f: CACTGCTGCTGAGATGAATGAAA, GM-CSF_r: GTCTGTAGGCAGGTCGGCTC, TNF-α_f GGAGAAGGGTGACCGACTCA, TNF-α_r: CTGCCCAGACTCGGCAA, GAPDH_f: TGCACCACCAACTGCTTAGC, and GAPDH_r: GGCATGGACTGTGGTCATGAG. Data were analyzed using GraphPad Prism 5 software (GraphPad Software, La Jolla, Calif., USA). All experiments were carried out in triplicate.

### EXAMPLE 1

IgG4 Reduces the Capture of Anti-TNF-α mAbs but This is Mediated by Blocking CD16

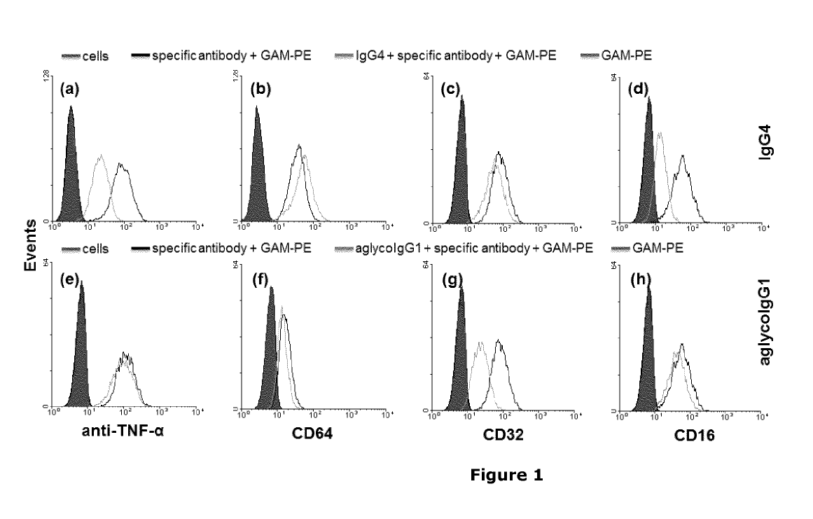

Human IgG1-Fcγ fragments have been shown to block CD64, thus rescuing the inhibitory activity of anti-TNF-α mAbs (Wojtal, K. A., et al. Fc gamma receptor CD64 modulates the inhibitory activity of infliximab. PLoS One 2012. 7: e43361). However, these fragments also block CD16 and CD32, which mediate important components of the primary immune response. In contrast to human IgG1, human IgG4 has a lower affinity towards CD16 and CD32 than towards CD64 (Bruhns, P., et al., Specificity and affinity of human Fcgamma receptors and their polymorphic variants for human IgG subclasses. Blood 2009, 113: 3716-3725). Human unrelated IgG4 mAbs was used to preferentially block CD64 while retaining most of the activity of CD16 and CD32. IgG4 was able to reduce the capture of anti-TNF-α mAbs substantially, but in contrast to our expectations this effect was mediated by CD16 rather than CD64 or CD32 (FIG. 1 upper panel). A human aglycoIgG1 mAb was used to specifically block CD64 because the loss of glycans has previously been shown to abolish CD16 and CD32 binding but to retain residual affinity towards CD64 (Jung, S. T., et al., Bypassing glycosylation: engineering aglycosylated full-length IgG antibodies for human therapy. Curr Opin Biotechnol 2011. 22: 858-867). Interestingly, the pre-incubation of IFN-γ-stimulated HL-60 cells with aglycoIgG1 resulted in the blocking of CD32, but not CD64 or CD16, However, this did not reduce the capture of anti-TNF-α mAbs, suggesting that only CD64 and CD16 are involved in this process (FIG. 1 lower panel).

### EXAMPLE 2

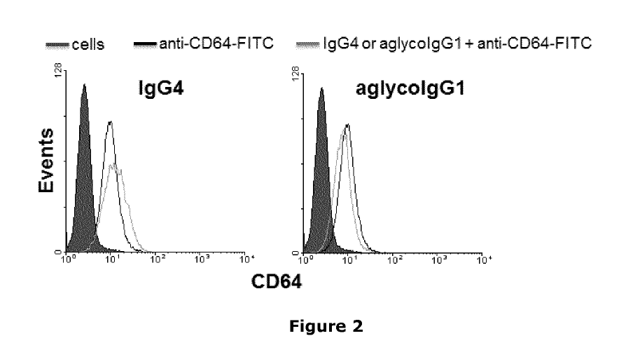

Serum has no impact on the binding of IgG4 and aglycoIgG1 to CD64 CD64 has a high affinity (10⁻⁷-10⁻⁸ M) for the Fcγ part of IgG molecules (Bruhns, P., et al., Specificity and affinity of human Fcgamma receptors and their polymorphic variants for human IgG subclasses. Blood 2009, 113: 3716-3725). in the presence of serum, the on-off-rate for CD64 and its ligands is therefore brief, which leads to near permanent engagement of the receptor by IgGs. One potential explanation for the inability of CD64 to bind IgG4 and aglycoIgG1 (as shown above) is the presence of IgGs in the serum-containing medium used for the cultivation of HL-60 cells. Therefore, HL-60 cells were cultivated in serum-free medium for 2 weeks and the flow cytometry experiments were repeated. However, neither IgG4 nor aglycoIgG1 bound to CD64 even in the absence of serum (FIG. 2).

### EXAMPLE 3

Generation and Analysis of the CD64-Specific Antibody Fragment H22(scFv)

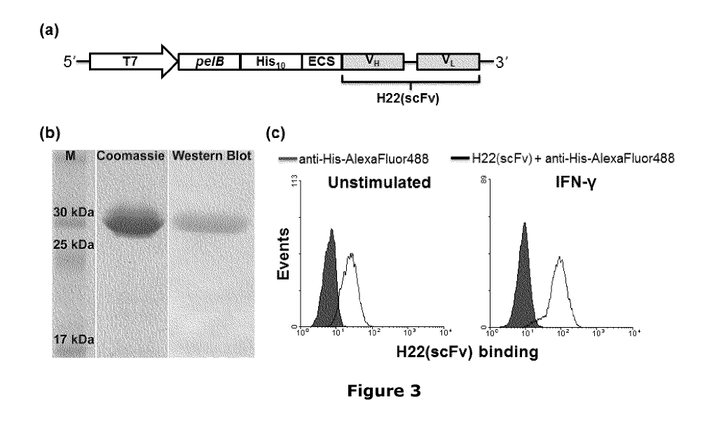

A single chain antibody fragment was generated named H22(scFv) that binds specifically to CD64. The H22(scFv) coding sequence was fused in frame with an N-terminal pelB leader peptide and a His₁₀ tag (FIG. 3a). The protein was successfully expressed in Escherichia coli using the periplasmic stress expression protocol (Barth, S., et al., Compatible-solute-supported periplasmic expression of functional recombinant proteins under stress conditions. Appl Environ Microbiol 2000. 66: 1572-1579). A yield of 0.14 mg purified protein/g bacterial pellet was obtained. The identity of purified H22(scFv) was confirmed by SDS-PAGE followed by staining the gel with Coomassie Brilliant Blue, and by western blot using polyhistidine-specific antibodies (FIG. 3b). In addition, flow cytometry showed that H22(scFv) specifically binds to CD64⁺ target cells (FIG. 3c), with no binding to CD64⁻L540cy cells (data not shown).

### EXAMPLE 4

The Reduced Capture of Anti-TNF-α mAbs Correlates Quantitatively With CD64-Specific Blocking by H22(scFv)

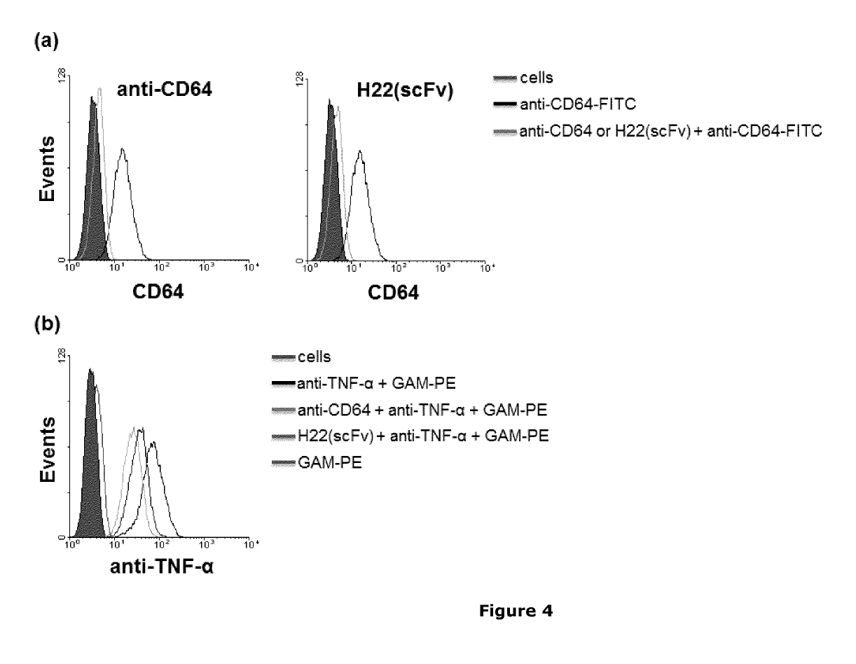

Recombinant H22(scFv) was used to specifically block CD64 on the surface of HL-60 cells, before adding the anti-TNF-α antibodies. Flow cytometry showed that H22(scFv) was able to block CD64 (FIG. 4a), thus reducing the capture of anti-TNF-α mAbs, and that the effect was comparable to that of a full-length anti-CD64 antibody (FIG. 4b).

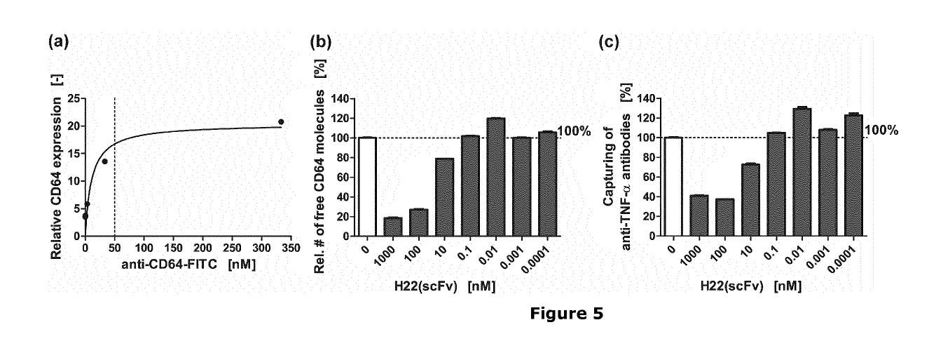

CD64 blocking was quantified by measuring the concentration of anti-CD64 mAb required to saturate all CD64 molecules on the cell surface, revealing a minimum saturating concentration of ≧50 nM (FIG. 5a). Because H22(scFv) is monovalent whereas the full-length anti-CD64 mAb is bivalent, 100 nM of H22(scFv) must theoretically be sufficient to block all CD64 molecules. This was confirmed by directly titrating H22(scFv) against cultured HL-60 cells (FIG. 5b), and we found that blocking CD64 with H22(scFv) quantitatively correlated with the reduced capture of anti-TNF-α mAbs (FIG. 5c).

### EXAMPLE 5

H22(scFv) Binding Does Not Induce Pro-Inflammatory Downstream Signaling

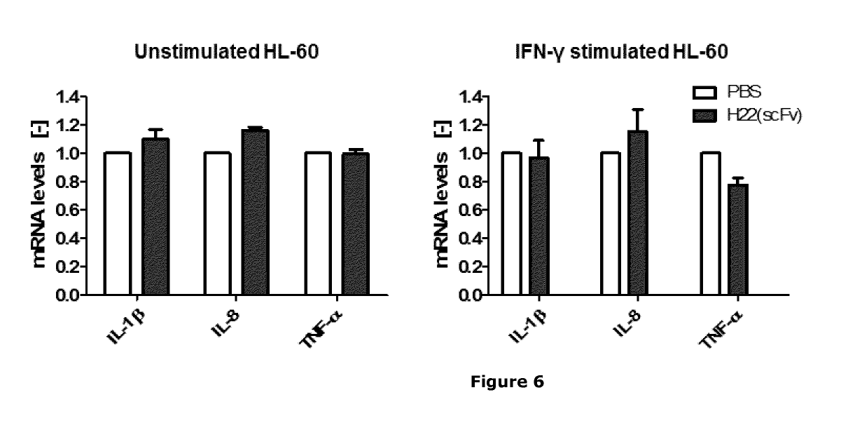

The Fcγ part of anti-TNF-α mAbs can induce the expression of pro-inflammatory genes when it binds to CD64, including those encoding TNF-α, IL-1β, IL-6, IL-8, and GM-CSF (Wojtal, K. A., et al. Fc gamma receptor CD64 modulates the inhibitory activity of infliximab. PLoS One 2012. 7: e43361). To test whether H22(scFv) also induces downstream cytokine production, unstimulated and IFN-γ-stimulated HL-60 cells were incubated with 100 nM H22(scFv) or anti-TNF-α mAb, or were pre-incubated with H22(scFv) and then supplemented with anti-TNF-α mAb. IFN-γ-stimulated HL-60 cells without additional reagents were used as a control. The induction of cytokine gene expression was measured by real-time PCR using gene-specific primers. Although cytokine genes were induced when the cells were incubated with anti-TNF-α mAbs alone, there was no induction in the presence of H22(scFv) alone and the ability of anti-TNF-α mAbs to induce pro-inflammatory cytokines was strongly attenuated by pre-incubation with H22(scFv) (FIG. 6). Poor responses to anti-TNF-α therapy in some patients are associated with the overexpression of CD64, which leads to the capture of anti-TNF-α mAbs and the induction of a pro-inflammatory response (Wojtal, K. A., et al. Fc gamma receptor CD64 modulates the inhibitory activity of infliximab. PLoS One 2012. 7: e43361). Blocking CD64 with H22(scFv) reduced the capture of anti-TNF-α mAbs, thus also limiting the downstream pro-inflammatory effects.

### EXAMPLE 6

The Expression of mTNF-α is Upregulated in IFN-γ-Stimulated HL-60 Cells

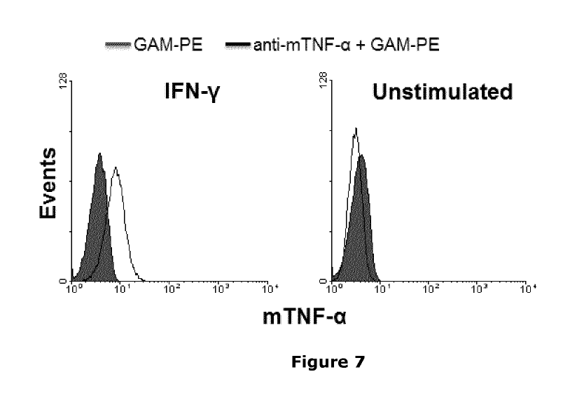

TNF-α exists as both a soluble cytokine and as a transmembrane protein, the latter lacking the systemic inflammatory effects of its soluble counterpart. Because inflammatory CD64^{high} monocytes/macrophages are likely to express high levels of mTNF-α, the capture of anti-TNF-α mAbs would be increased via their antigen binding Fab domain. IFN-γ-stimulated (pro-inflammatory) and non-stimulated HL-60 cells were tested for the expression of mTNF-α using a mAb that selectively recognizes the transmembrane form (Gerspach, J.,et al., M., Detection of membrane-bound tumor necrosis factor (TNF): an analysis of TNF-specific reagents. Microsc Res Tech 2000. 50: 243-250). It was found that only the stimulated HL-60 cells expressed mTNF-α (FIG. 7), indicating the existence of an additional mechanism to capture anti-TNF-α mAbs.

### EXAMPLE 7

The Capture of Anti-TNF-α mAbs is Mediated by CD64 and mTNF-α

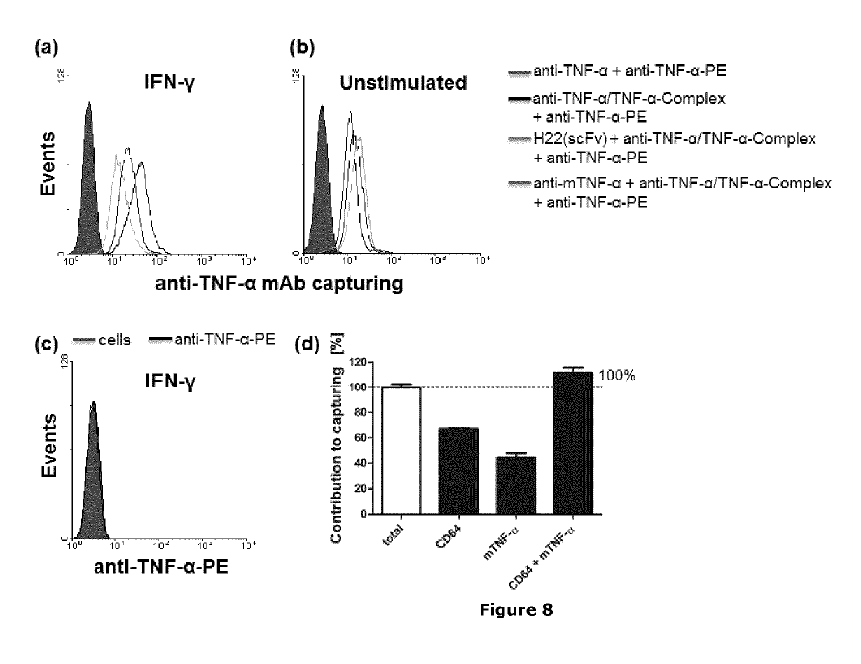

To determine whether mTNF-α is involved in the capture of anti-TNF-α mAbs, mTNF-α was pre-blocked on the surface of stimulated HL-60 cells using the mTNF-α-specific antibody described above, before measuring the capacity of the cells to capture anti-TNF-α mAbs. Blocking mTNF-α reduced the ability of the cells to capture anti-TNF-α mAbs as expected (FIG. 8a-c), and the additive effect of blocking by H22(scFv) and anti-mTNF-α was found to match the total capacity of stimulated HL-60 cells to capture anti-TNF-α mAbs (FIG. 8d). This result confirms that the capture of anti-TNF-α mAbs is dependent on the expression of both CD64 and mTNF-α.

1. A polypeptide having a binding affinity to CD64 of at least 10⁻⁹ M for use in a treatment of a disease associated with an inflammation in combination with an anti-TNF-α antibody.

2. The polypeptide of claim 1 wherein the disease associated with an inflammation is selected from the group consisting of inflammatory bowel disease, rheumatoid arthritis, multiple sclerosis, lupus, sclerodoma; psoriasis; gastrointestinal, graft-versus-host-disease, Crohn's disease, diabetes, ulcerative colitis, acute and chronic transplantation rejection, colitis, bronchiolitis obliterans, ileitis, pancreatitis, glomerulonephritis, uveitis, arthritis, hepatitis, dermatitis, artherosclerosis, all forms of arthritis, including, but not only Rheumatoid arthritis, Multiple sclerosis, ankylosing spondylitis, hidradenitis suppurativa and atopic dermatitis.

3. The polypeptide of claim 1 wherein the polypeptide is an antibody.

4. The polypeptide of claim 1 wherein the polypeptide is an antibody fragment.

5. The polypeptide of claim 4 wherein the antibody fragment is encoded by a polynucleotide of SEQ ID NO: 1.

6. The polypeptide of claim 4 wherein the antibody fragment is a recombinant polypeptide.

7. The polypeptide of claim 4 having the amino acid sequence of SEQ ID NO: 2.

8. (canceled)

9. A mixture comprising the polypeptide of claim 1 and a blocking agent of transmembrane TNF-α and/or soluble TNF-α.

10. A method of treating a disease associated with an inflammation comprising administering to a patient in need thereof a polypeptide of claim 1 in combination with an anti-TNF-α antibody.

11. The method of claim 10 wherein the patient is a non-responder to a treatment of inflammation in a therapy employing an anti-TNF-α antibody.

12. A method for enhancing the therapy of a patient in a therapy employing an anti-TNF-α antibody comprising administering to a patient in need thereof a polypeptide of claim 1 in combination with an anti-TNF-═ antibody.

13. The method of claim 12 wherein the patient is a non-responder to a treatment of inflammation in a therapy employing an anti-TNF-α antibody.

14. The method of claim 10 wherein the polypeptide is administered combination with a blocking agent of transmembrane TNF-α.


SEQUENCE LISTING (14 sequences)

SEQ ID NO: 1 - DNA, 747 bp, homo sapiens
atggcccaggtgcagctggtggagagcggtggaggtgttgtgcaacctggccggtccctgcgcctgtcctgctcctcgtctggcttcattttcagtgacaattacatgtattgggtgagacaggcacctggaaaaggtcttgagtgggttgcaaccattagtgatggtggtagttacacctactatccagacagtgtgaagggaagatttacaatatcgagagacaacagcaagaacacattgttcctgcaaatggacagcctgagacccgaagacaccggggtctatttttgtgcaagaggctactataggtacgagggggctatggactactggggccaagggaccccggtcaccgtgagctcaggaggtggcggctccggaggtggaggcagcggagggggcggatccgacatccagctgacccagagcccaagcagcctgagcgccagcgtgggtgacagagtgaccatcacctgtaagtccagtcaaagtgttttatacagttcaaatcagaagaactacttggcctggtaccagcagaagccaggtaaggctccaaagctgctgatctactgggcatccactagggaatctggtgtgccaagcagattcagcggtagcggtagcggtaccgacttcaccttcaccatcagcagcctccagccagaggacatcgccacctactactgccatcaatacctctcctcgtggacgttcggccaagggaccaagctggagatcaaa

SEQ ID NO: 2 - protein, 278 aa, artificial
  recombinant antibody fragment
HHHHHHHHHHSSGHIDDDDKHMKLMAQPAMAQVQLVESGGGVVQPGRSLRLSCSSSGFIFSDNYMYWVRQAPGKGLEWVATISDGGSYTYYPDSVKGRFTISRDNSKNTLFLQMDSLRPEDTGVYFCARGYYRYEGAMDYWGQGTPVTVSSGGGGSGGGGSGGGGSDIQLTQSPSSLSASVGDRVTITCKSSQSVLYSSNQKNYLAWYQQKPGKAPKLLIYWASTRESGVPSRFSGSGSGTDFTFTISSLQPEDIATYYCHQYLSSWTFGQGTKLEIK

SEQ ID NO: 3 - DNA, 21 bp, artificial
  primer
acagatgaagtgctccttcca

SEQ ID NO: 4 - DNA, 21 bp, artificial
  primer
gtcggagattcgtagctggat

SEQ ID NO: 5 - DNA, 21 bp, artificial
  primer
ggtacatcctcgacggcatct

SEQ ID NO: 6 - DNA, 21 bp, artificial
  primer
gtgcctctttgctgctttcac

SEQ ID NO: 7 - DNA, 23 bp, artificial
  primer
actgagagtgattgagagtggac

SEQ ID NO: 8 - DNA, 21 bp, artificial
  primer
aaccctctgcacccagttttc

SEQ ID NO: 9 - DNA, 23 bp, artificial
  primer
cactgctgctgagatgaatgaaa

SEQ ID NO: 10 - DNA, 20 bp, artificial
  primer
gtctgtaggcaggtcggctc

SEQ ID NO: 11 - DNA, 20 bp, artificial
  primer
ggagaagggtgaccgactca

SEQ ID NO: 12 - DNA, 17 bp, artificial
  primer
ctgcccagactcggcaa

SEQ ID NO: 13 - DNA, 20 bp, artificial
  primer
tgcaccaccaactgcttagc

SEQ ID NO: 14 - DNA, 21 bp, artificial
  primer
ggcatggactgtggtcatgag